(19) United States
(12) Patent Application Publication
(10) Pub. No.: US 2008/0003696 A1
(43) Pub. Date: Jan. 3, 2008

(54) Embossed reagent card

(76) Inventor: Andrew Rae, Toronto (CA)

Correspondence Address: Anthony C. Kuhlmann, Ph.D.;Brown Rudnick Berlack Israels LLP, One Financial Center, Box IP, Boston, MA, 02111, US

(21) Appl. No.: 11/479,089

(22) Filed: Jun. 30, 2006

(51) Int. Cl. G01N 33/558

(52) U.S. Cl. 436/514

(57) ABSTRACT

This invention features a reagent card suitable for performing small volume colorimetric and other visually-readable reactions. The reagent card is characterized by one or more liquid containment wells defined by an embossed perimeter.

### FIELD OF THE INVENTION

This present disclosure relates to laboratory test equipment for performing small volume colorimetric and other visually-readable reactions.

### BACKGROUND OF THE INVENTION

There exist a variety of clinical, epidemiological, and microbiological tests that are designed to be rapidly performed and visually interpreted by the observer (i.e., without the need for specialized detection equipment). These tests range from blood type agglutination assays to environmental pathogen and other microbiological detection tests. These assays are performed in small volumes (>2 ml) and the readouts are frequently colorimetric or agglutination-based indicators. It is most desirable that the test equipment is portable, disposable, and inexpensive.

### SUMMARY OF THE INVENTION

The present invention is an improved assay reagent card. The card is characterized by one or more (e.g., 2, 4, 6, 8, 10, or more) liquid containment wells which are formed by a embossed (raised) perimeter. The wells are designed to hold any convenient volume of reagents, but typically holds about 100 μl, 250 μl, 500 μl, 750 μl, 1 ml, 1.25 ml, 1.5 ml, 2.0 ml, or more. Likewise, the embossed perimeter is any convenient height above the plane of the reagent card. Typically, the perimeter is about 0.50 mm, 0.75 mm, 1.0 mm, 1.5 mm, 2.0 mm or more in height.

The reagent card is made of any convenient material that is compatible with the specific reagents for each contemplated application or assay. Useful materials for constructing the reagent cards include, for example, paperboard (plastic coated or uncoated), latex, polypropylenes, polyethylenes (e.g., polyethylene terephthalate), and the like. The reagent card may be a single ply or a laminate structure. For reagent cards having a laminate structure, each layer may be the same or a different material.

In useful embodiments, the embossed perimeter may be created during or after the formation of the reagent card (e.g., by pressing a form into the back of the reagent card to create the embossed perimeters having a positive relief on the upper surface) or by laminating a perimeter form between a top layer and a bottom layer of a laminate reagent card. The liquid containment wells may be any convenient shape, but are typically circular.

The color of the reagent card or the liquid containment wells may be any useful color and the color is varied based on the particular assay endpoint. The color is chosen to enhance the contrast between the assay card (i.e., the bottom of the containment well) and the assay solution that will be used. Common color choices include white, black, and blue, but any suitable color may be used. The reagent card may be any convenient size depending upon the size, shape, and number of liquid containment wells desired.

Typically, reagent cards of this invention have a length dimension and a width dimension each of about 2″-10″. In a particularly useful embodiment, the reagent card has a dimension of about 2.5″×5.0″ with eight circular liquid containment wells each having a diameter of about 1″.

### BRIEF DESCRIPTION OF DRAWINGS

The foregoing and other features and advantages of the present disclosure will be more fully understood from the following detailed description of the exemplary embodiments, taken in conjunction with the accompanying drawings in which:

FIG. 1 is a perspective view of the upper surface of a reagent card constructed in accordance with the principles of the present disclosure;

FIG. 2 is a lateral view of the long edge of a reagent card constructed in accordance with the principles of this disclosure;

FIG. 3 is a plan view of a reagent card constructed in accordance with the principles of this disclosure;

FIG. 4 is a lateral view of the short edge of a reagent card constructed in accordance with the principles of this disclosure;

FIG. 5 is a plan view of the lower surface of a reagent card constructed in accordance with the principles of this disclosure; and

FIG. 6 is a perspective view of a laminated reagent card showing the perimeter form located within the laminate structure.

### DETAILED DESCRIPTION

The present invention is an assay reagent card having one or more liquid containment wells that are formed by an embossed perimeter. The embossed perimeter that forms the liquid containment wells is a significant improvement over currently available test equipment.

The reagent cards of this invention are used for low volume, visually interpreted assays. Often, these low volume, visually-readable assays are performed on disposable reagent cards or glass slides. Some disposable reagent cards usually have multiple printed circles and varying background colors, depending upon the application. The printed circles provide a “target” area for the user to place the assay reagents. These target areas help the user organize multiple assays on a single disposable card and to run the assays side-by-side for comparison purposes. The flat reagent cards have the disadvantage that the reagents are held in place only by the surface tension of the liquid, requiring that the reagent cards be kept flat and relatively motionless.

Another method for performing these types of assays involves the use of glass slides similar to microscope slides. The problem of containing the reagents is overcome through the use of a commercially available “hydrophobic pen”. These pens dispense a hydrophobic gel that adheres to the glass slide, forming a hydrophobic barrier that aids in reagent containment. The disadvantage of the hydrophobic barrier method is that the barrier height is limited by the amount of hydrophobic gel that can be released in a single pass by the pen. Layering is difficult because application of an upper layer frequently causes the detachment of the lower layers of gel from the slide surface. Also, many assays use reagents that contain detergents which are capable of detaching, solubilizing, or otherwise breaching the hydrophobic barrier.

The present invention solves the disadvantages of these known methods. An embossed reagent card provides a physical barrier to contain the individual assays and the barrier is physio-chemically inert to the assay reagents. The embossed reagent card also eliminates the requirement of the user to “draw” a containment well for each assay. The size uniformity of the embossed liquid containment wells is also desirable when reading qualitative visual results in which the quality or magnitude of a color change is a significant end point. For example, a fixed volume of a colored solution appears less intensely colored when spread over a larger surface area.

Turning now to the figures, wherein like components are designated by like reference numerals throughout the several views. FIGS. 1 and 3 illustrate one embodiment of the reagent card 10 constructed in accordance with the principles of this disclosure. The reagent card 10 has a plurality (eight, in this case) liquid containment wells 12 that are formed by embossed perimeters 14. The embossed perimeters 14 are raised above the plane of the upper surface 19 of the reagent card 10. Each liquid containment well 12 is intended to contain a single assay.

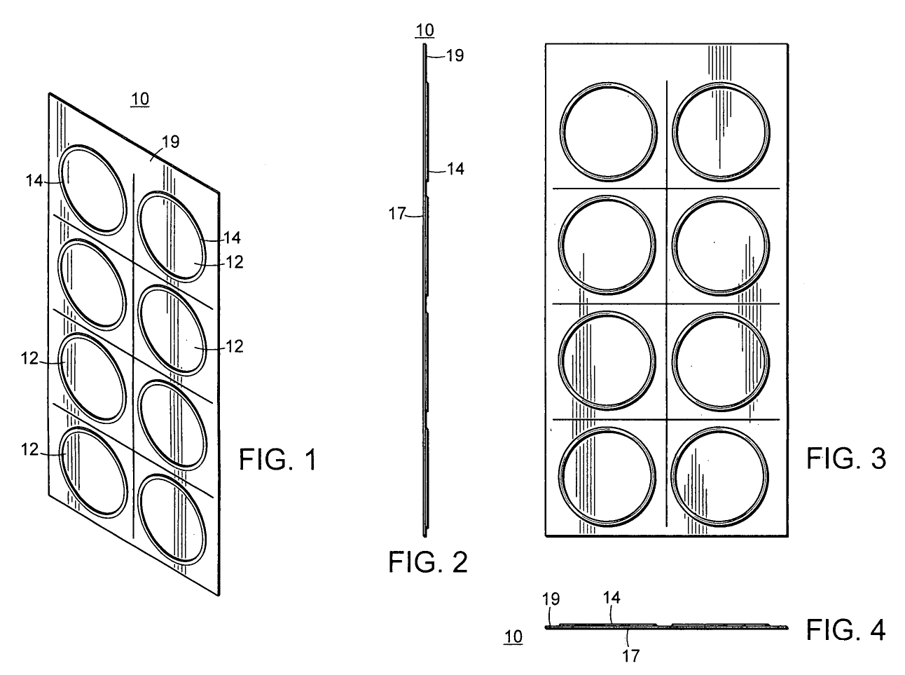

FIGS. 2 and 4 are perspective views from the edges of the reagent card 10. The embossed perimeters 14 are raised above the plane of the upper surface 19. The lower surface 17 of the reagent card 10 is substantially flat. In some embodiments, the lower surface 17 features a negative relief 21 (not shown; see FIG. 5) of the embossed perimeters 14.

FIG. 5 is a plan view of the lower surface 17 of the reagent card 10. In one embodiment, the lower surface 17 features negative relief 21 of the embossed perimeters 14. Typically, the embossed perimeters 14 (not shown) are created on the upper surface 19 by making a positive impression on the lower surface 17, thereby creating vertical relief on the upper surface 19 and leaving the negative relief 21 (i.e., grooves) in the lower surface 17. In alternative embodiments, the embossed perimeters 14 are created during the molding process, resulting in a smooth and flat lower surface 17 devoid of negative relief 21.

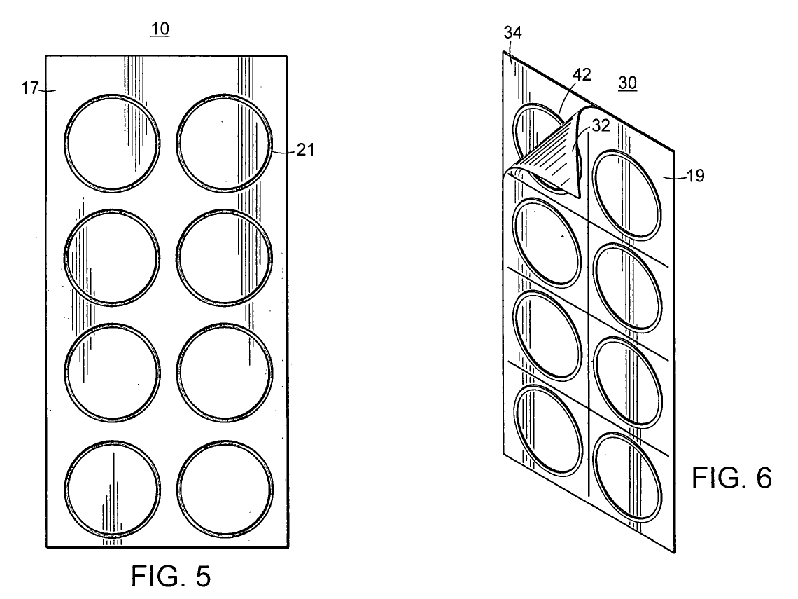

FIG. 6 is a perspective view of a laminate reagent card 30 constructed in accordance with the principles of this invention. The reagent card 30 is a laminate structure constructed of any suitable material or combination of materials (e.g., paperboard, latex, polyethylene, polypropylene, etc.). The materials are selected based on the intended assay and chemical reagents that will be used and the physical properties of those materials. The laminate reagent card 30 consists of at least a top layer 32 and a bottom layer 34, but may include additional laminate layers as desired for particular applications. A perimeter form 42 is sealed between the top layer 32 and the bottom layer 34 such that the perimeter form 42 causes vertical relief on the upper surface 19 of the reagent card 30. In preferred embodiments, the bottom layer 34 remains flat (i.e., devoid of relief). The perimeter form 42 is sealed between the top layer 32 and the bottom layer 34 by any convenient method including, for example, heat sealing or thermal melting. The perimeter form 42 is made of any convenient material including, for example, metal or a thermoplastic or other polymer provided that the form 42 maintains its shape during the manufacturing process.

### Other Embodiments

Although the foregoing invention has been described in some detail by way of illustration and example for purposes of clarity of understanding, it will be readily apparent to those of ordinary skill in the aft in light of the teachings of this invention that certain changes and modifications may be made thereto without departing from the spirit or scope of the appended claims.

What is claimed is:

1. A reagent card comprising one or more liquid containment wells, wherein each of said liquid containment wells is defined by an embossed perimeter.

2. The reagent card of claim 1, wherein said each of said liquid containment wells has a volume of about 100 μl-1 ml.

3. The reagent card of claim 2, wherein each of said liquid containment wells has a volume of about 0.5 ml.

4. The reagent card of claim 2, wherein each of said liquid containment wells is circular.

5. The reagent card of claim 1, wherein said reagent card is a single ply.

6. The reagent card of claim 1, wherein said reagent card has a laminate structure.

7. The reagent card of claim 1, wherein said reagent card comprises latex or paperboard.

8. The reagent card of claim 1, wherein said embossed perimeters have a height of about 0.5 mm-2.0 mm.

9. The reagent card of claim 8, wherein said embossed perimeters have a height of about 1.0 mm.

10. The reagent card of claim 1, wherein said reagent card comprises 4-10 liquid containment wells.

11. A reagent card comprising at least one ply of latex and 4-10 circular liquid containment wells, wherein each of said liquid containment wells is defined by an embossed perimeter and has a volume of 100 μl-1 ml.

12. The reagent card of claim 11, wherein said reagent card comprises 8 liquid containment wells.

13. The reagent card of claim 12, wherein each of said liquid containment wells has a volume of about 0.5 ml.

14. The reagent card of claim 13, wherein each of said embossed perimeters has a height of about 1.0 mm.
